(19) United States
(12) Patent Application Publication
(10) Pub. No.: US 2026/0142003 A1
(43) Pub. Date: May 21, 2026

(54) Clinical Symptom Analysis

(71) Applicant: REGENTS OF THE UNIVERSITY OF MICHIGAN, Ann Arbor, MI (US)

(72) Inventors: Sekhar Chandra Sripada, Ann Arbor, MI (US); Aman Taxali, South Lyon, MI (US); Keith Michael Angstadt, Milan, MI (US)

(21) Appl. No.: 19/393,022

(22) Filed: Nov. 18, 2025

Related U.S. Application Data
(60) Provisional application No. 63/721,940, filed on Nov. 18, 2024.

(51) Int. Cl. G16H 15/00; G16H 50/20

(52) U.S. Cl. CPC G16H 15/00; G16H 50/20

(57) ABSTRACT

The following relates generally to improved clinical symptom analysis. In some embodiments, one or more processors: (1) receive patient input data; (2) obtain patient survey data representing the patient's condition; (3) determine a trend in the patient survey data using a determination algorithm; (4) apply the generative AI algorithm to the patient input data, the patient survey data, and the trend in the patient survey data to produce a report of the trend of the patient's condition; and/or (5) display the report on a display device.

### CROSS REFERENCE TO RELATED APPLICATIONS

This application claims the benefit of U.S. Provisional Application No. 63/721,940, entitled “Clinical Symptom Analysis” (filed Nov. 18, 2024), the entirety of which is incorporated by reference herein.

### FIELD

The present disclosure generally relates to improved clinical symptom data collection and summarization using generative artificial intelligence (AI).

### BACKGROUND

Psychiatric clinical care providers need to know what symptoms and stressors the patient has been experiencing in the weeks before the clinical visit. The main current method is through asking the patient to recall and report what has been happening in the previous few weeks, but this creates many challenges. For example, during monthly meetings with a psychiatric clinical care provider, the patient may focus her discussion on more recent events and not mention important information from less recent events. In another example, the psychiatric clinical care provider may spend valuable time attempting to determine what happened between sessions instead of spending time addressing the patient's issues.

The systems and methods disclosed herein may provide solutions to these problems and may provide solutions to the ineffectiveness, difficulties, inefficiencies, encumbrances, and/or other drawbacks of conventional recall-based techniques.

### SUMMARY

The following relates generally to presenting a report of a patient's condition. More specifically, the following uses artificial intelligence to summarize a patient's condition from patient input over time and then provides that summary to the healthcare provider on demand in order to reveal ongoing issues the patient may not recall.

In one aspect, a computer-implemented method for presenting a report of a patient's condition may be provided. The method may include: (1) receiving, by one or more processors, patient input data (for example, daily voice recordings); (2) obtaining, by the one or more processors, patient survey data representing the patient's condition; (3) determining, by the one or more processors, a trend in the patient survey data using a determination algorithm; (4) applying, by the one or more processors, the generative AI algorithm to the patient input data, the patient survey data, and the trend in the patient survey data to produce a report of the trend of the patient's condition; and/or (5) displaying, by the one or more processors, the report on a display device. The method may include additional, fewer, or alternate actions, including those discussed else-where herein.

In another aspect, a computer system for presenting a report of a patient's condition may be provided. The computer system may include one or more processors configured to: (1) receive patient input data (for example, daily voice recordings); (2) obtain patient survey data representing the patient's condition; (3) determine a trend in the patient survey data using a determination algorithm; (4) apply the generative AI algorithm to the patient input data, the patient survey data, and the trend in the patient survey data to produce a report of the trend of the patient's condition; and/or (5) display the report on a display device. The computer system may include additional, less, or alternate functionality, including that discussed elsewhere herein.

In yet another aspect, a computing device for presenting a report of a patient's condition may be provided. The computing device may include one or more memories and/or one or more processors. The one or more memories may have stored thereon computer-executable instructions that, when executed by the one or more processors, cause the computing device to: (1) receive patient input data (for example, daily voice recordings); (2) obtain patient survey data representing the patient's condition; (3) determine a trend in the patient survey data using a determination algorithm; (4) apply the generative AI algorithm to the patient input data, the patient survey data, and the trend in the patient survey data to produce a report of the trend of the patient's condition; and/or (5) display the report on a display device. The computing device may include additional, less, or alternate functionality, including that discussed elsewhere herein.

### BRIEF DESCRIPTION OF THE DRAWINGS

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

The figures described below depict various aspects of the applications, methods, and systems disclosed herein. It should be understood that each figure depicts an embodiment of a particular aspect of the disclosed applications, systems and methods, and that each of the figures is intended to accord with a possible embodiment thereof. Furthermore, wherever possible, the following description refers to the reference numerals included in the following figures, in which features depicted in multiple figures are designated with consistent reference numerals.

FIG. 1 shows an example system for presenting a report of a patient's condition, according to some aspects.

FIG. 2 shows an example method for presenting a report of a patient's condition, according to some aspects.

FIG. 3 shows and example method for presenting a report of a patient's condition based on an interval, according to some aspects.

FIG. 4 shows and example screen for prompting a patient for input, according to some aspects.

FIG. 5 shows an example screen for prompting a patient for survey input, according to some aspects.

FIG. 6 shows an example of a screen for displaying a report of the patient's condition, according to some aspects.

FIG. 7 shows an example of a screen for displaying a report of the patient's condition, according to some aspects.

FIG. 8 shows an example of a screen for displaying a report of the patient's condition along with citations and annotations, according to some aspects.

FIG. 9 shows an example of selecting a date range to generate a report.

FIG. 10 shows and example of selecting date ranges for an interval change report.

FIG. 11 shows an example combined block and logic diagram for training an example chatbot model.

### DETAILED DESCRIPTION

The present embodiments relate to, inter alia, presenting a report on a patient's condition based on data collected from the patient in between sessions with their clinician. For example, a patient may verbalize into a recording device daily their thoughts and feelings regarding their day along with answering a survey designed to place numerical values on how their day went. The record along with the survey data may be used together by the generative AI to create a summary. Advantageously, this improves the quality of the information available to the clinician. For example, during monthly meetings, the patient may have forgotten less recent events (e.g., events from 3 and a half weeks ago, etc.), over-emphasize more recent events, etc. Embodiments described herein address this challenge and others.

### Example System

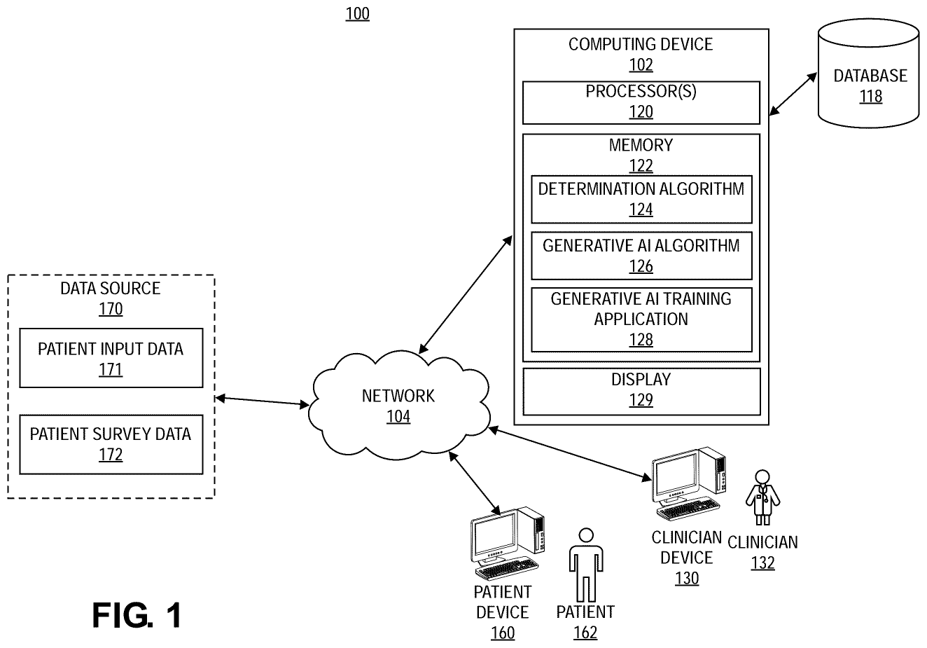

FIG. 1 shows an example system 100 for presenting a report on a patient's condition. The high-level architecture illustrated in FIG. 1 may include both hardware and software applications, as well as various data communications channels for communicating data between the various hardware and software components, as is described below. The system may include a computing device 102 configured to communicate (e.g., via a network 104, which may be a wired or wireless network, such as the Internet), with a clinician device 130, patient device 160, and/or data source 170.

The computing device 102 may include one or more processors 120 such as one or more microprocessors, controllers, and/or any other suitable type of processor (e.g., one or more central processing units (CPUs), one or more graphics processing units (GPUs), etc.). The computing device 102 may further include a memory 122 (e.g., volatile memory, non-volatile memory) accessible by the one or more first processors 120 (e.g., via a memory controller). Additionally, the computing device 102 may include a display 129. In some embodiments, the computing device 102 is part of a cloud computing platform.

The one or more processors 120 may interact with the memory 122 to read and to execute computer-readable instructions stored in the memory 122. Additionally or alternatively, computer-readable instructions may be stored on one or more removable media (e.g., a compact disc, a digital versatile disc, removable flash memory, etc.) that may be coupled to the computing device 102 to provide access to the computer-readable instructions stored thereon. In particular, the computer-readable instructions stored on the memory 122 may include one or more sets of instructions, such as a determination algorithm 124, a generative AI algorithm 126, and/or generative AI training application 128. More or fewer sets of instructions may be included, in some aspects.

The computing device 102 may be any suitable device. For example, the computing device 102 may be one or more servers, one or more personal computers, one or more smartphones, one or more tablets, one or more phablets, etc. It should be understood that although the example of FIG. 1 illustrates only one computing device 102, any number of computing devices 102 may be used.

The determination algorithm 124 may be a set of computer-executable instructions accessible by the one or more processors 120. The determination algorithm 124 may include computer-executable instructions that receive data from the data source 170 and process the data. For instance, as will be described in further detail below, the determination algorithm 124 may take data from the data source 170 from a range of dates to determine which days the patient 162 experienced better or worse days than normal.

The determination algorithm 124 may be a statistical algorithm, a deterministic algorithm, etc. Examples of statistical algorithms include: linear regression, clustering algorithm, classification algorithms, Bayesian algorithms, monte Carlo methods, etc. Examples of deterministic algorithms include: sorting algorithms, search algorithms, pathfinding algorithms, mathematical algorithms, greedy algorithms, dynamic programming algorithms, parsing algorithms, etc.

The generative AI algorithm 126 may include a set of computer-executable instructions accessible by the one or more processors 120. The generative AI algorithm 126 may include computer-executable instructions that use the results of the determination algorithm 124 to produce a report of a patient's condition, such as a summary of the patient's condition, summary of events on a particular day, or how one set of dates compare to another set of dates.

In some aspects, the computing device 102 may annotate the presented report with quotes or data from the data source 170.

The data that is used by the determination algorithm 124 and the generative AI algorithm 126 in order to create a report on a patient's condition may come from any suitable source. For example, the data may be sent to the computing device 102 from a data source 170. One example of the data held by the data source 170 includes patient input data 171, such as audio recordings, patient writings, patient video logs, etc. Another example of the data held by the data source 170 includes patient survey data 172, such as responses to survey questions that may be used to numerically represent how a patient's day went.

The determination algorithm 124 and generative AI algorithm 126 may also use data held by the database 118. An example of the database 118 includes a proprietary database owned by a company that also owns the computing device 102. Furthermore, it should be understood that although the example of FIG. 1 illustrates only one database 118, any number of databases 118 may be used.

In addition, data that is used to generate reports may also be received from the patient device 160 (e.g., a computing device, such as a smart phone, a personal computing device, a tablet, a phablet, a smart watch, a smart medical device, a medical monitor, etc.). In some examples, the patient device 160 belongs to a patient 162, for example, a patient being assessed by the computing device 102.

The generation of reports may be initiated by and/or viewed on the clinician device 130 (e.g., a computing device, such as a smart phone, a personal computing device, a tablet, a phablet, a smart watch, a smart medical device, a medical monitor, etc.). In some examples, the clinician device 130 belongs to a clinician 132 (e.g., doctors, nurses, healthcare providers, psychiatrists, psychologists, physician assistants, nurse practitioners, social workers, etc.), for example, a clinician using the computing device 102. In other embodiments, the clinician device 130 can perform the functions of the computing device 102.

Furthermore, it should be understood that the data may be received by the computing device 102 from more than one of the data sources 170, the patient device 160, and/or the database 118. Moreover, it should be understood that although the example of FIG. 1 illustrates only one of each of the clinician device 130, clinician 132, patient device 160 and patient 162, any number of clinician devices 130, clinicians 132, patient devices 160 and/or patients 162 may be used.

The data (e.g., held by any of the data sources 170, and/or the database 118) may include any data. Examples of the data include: audio recordings, video recordings, transcriptions of audio or video recordings, written entries, survey responses including but not limited to surveys using the Likert scale and/or other medical surveys. In addition, the data may be respective patient input data; that is, the patient input data corresponds to individual patients, and thus may be used to generate reports of that patient's condition.

Furthermore, it should be understood that although the example of FIG. 1 illustrates only one of each of the data sources 170, any number of data sources may be used (e.g., more than patient input data 171, more than one patient survey data 172, etc.).

### Example Methods

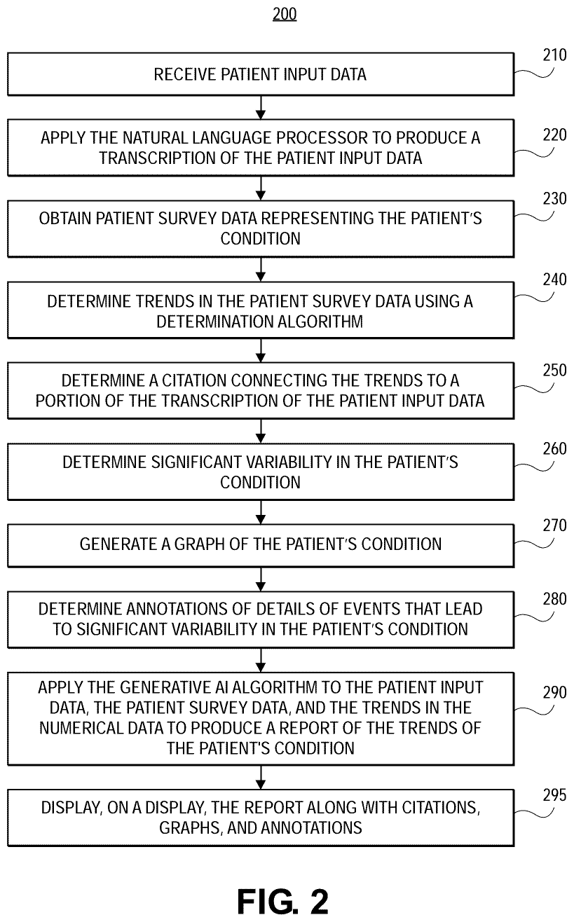

FIG. 2 shows an example method 200 for presenting a report on a patient's condition. For illustrative purposes, the following discussion will refer to the one or more processors 120 of the computing device 102 as performing the blocks of the example method 200. However, it should be understood that any other component (e.g., one or more processors of any of the data source 170, one or more processors of the clinician device 130, one or more processors of the patient device 160, etc.) may perform any of the blocks instead of and/or in conjunction with the one or more processors 120.

The example method 200 may include receiving patient input data (block 210). As discussed above, the patient input data 171 may be received from any suitable source, such as a data source 170, the patient device 160, and/or the database 118. As discussed above, examples of patient input data 171 include audio recordings, patient writings, patient video logs, etc.

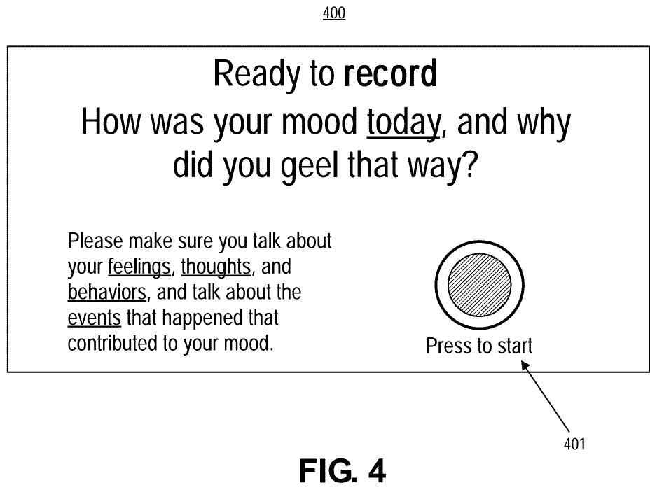

In some examples, the patient 162 may enter the patient input data 171 via example screen 400 of FIG. 4. The example screen 400 may include a patient input field 401. The patient input field 401 may be a button, text input box, or other element that prompts the patient 162 to interact with the patient input field 401. Interacting with the patient input field 401 allows the patient 162 to enter text, record their voice, record a video, or produce another type of record which may be stored on the patient device 160, data source 170, and/or a database 118.

The example method 200 may apply a natural language processor (NLP) to the patient input data 171 to produce a transcription of the patient input data 171 (block 220). The NLP may transform the patient input data (e.g., audio recordings, patient writings, patient video logs, etc.) to an interpretable format.

The example method 200 may obtain patient survey data 172 representing the patient's condition (block 230). The patient survey data 172 representing the patient's condition may be obtained from survey responses including but not limited to surveys using the Likert scale and other medical surveys, as discussed above. The patient survey data 172 representing the patient's condition may be received from any suitable source, such as a data source 170, the patient device 160, and/or the database 118.

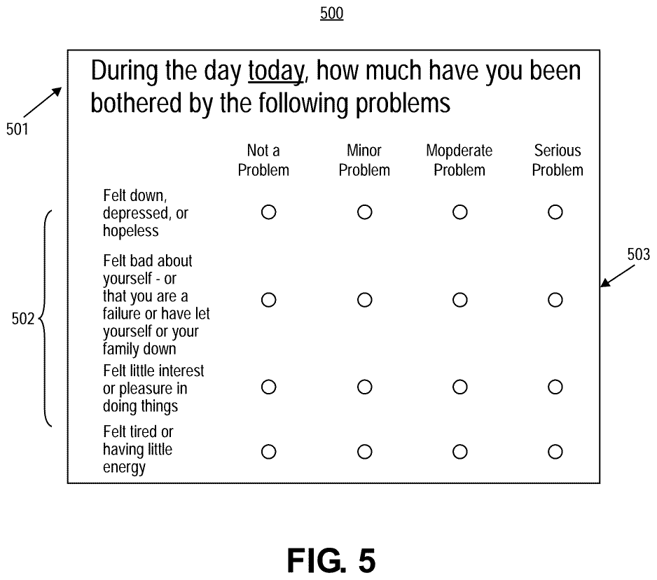

In some examples, the patient 162 may enter the patient survey data 172 via example survey 500 of FIG. 5. The example survey 500 may include a survey prompt 501, a list of survey questions 502, and/or a survey response field 503. The survey 500 may be a predetermined survey or a survey customized by the clinician 132 in order to assess conditions the patient 162 may be treated for. The example survey 500 shows a Likert scale survey, however the survey may be another survey such as: a numeric rating scale, a semantic differential scale, Guttman scale, hospital anxiety and depression scale, Beck depression inventory, generalized anxiety disorder 7-item scale, etc. The list of survey questions 502 may be a list of pre-generated questions related to the type of survey or customized or altered by the clinician 132. Although the example of FIG. 5 illustrates the survey response field 503 in multiple choice format, it should be appreciated that any suitable format may be used. For example, the survey may be presented as a series of buttons that, once clicked, answer the question, and trigger the presentation of a new question. In another example, answers to the questions may be entered by selecting a position on a slider bar. In yet another example, the survey may allow multiple answers to a single question. The example survey 500 may be one survey or a plurality of surveys.

Additionally, the example method 200 may use the patient survey data 172 obtained in block 230 to determine trends in the patient survey data 172 using a determination algorithm (block 240). The determination algorithm may be any algorithm that is capable of taking the objective data from patient surveys and returning data that can be used to signify a patient's condition. There may be a plurality of algorithms that work together in order to determine a patient's condition, or a plurality of algorithms of which one is selected either automatically or manually to be used.

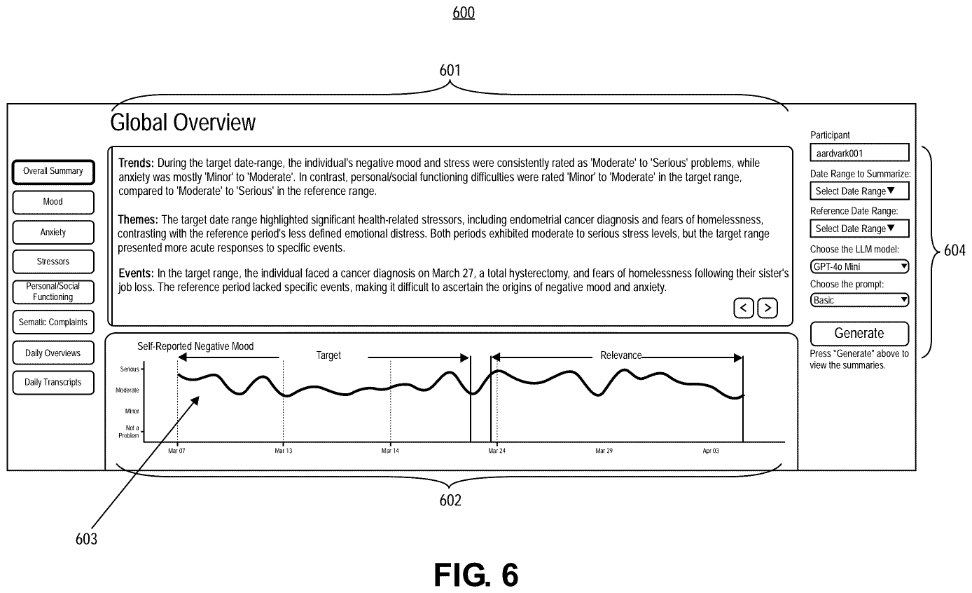

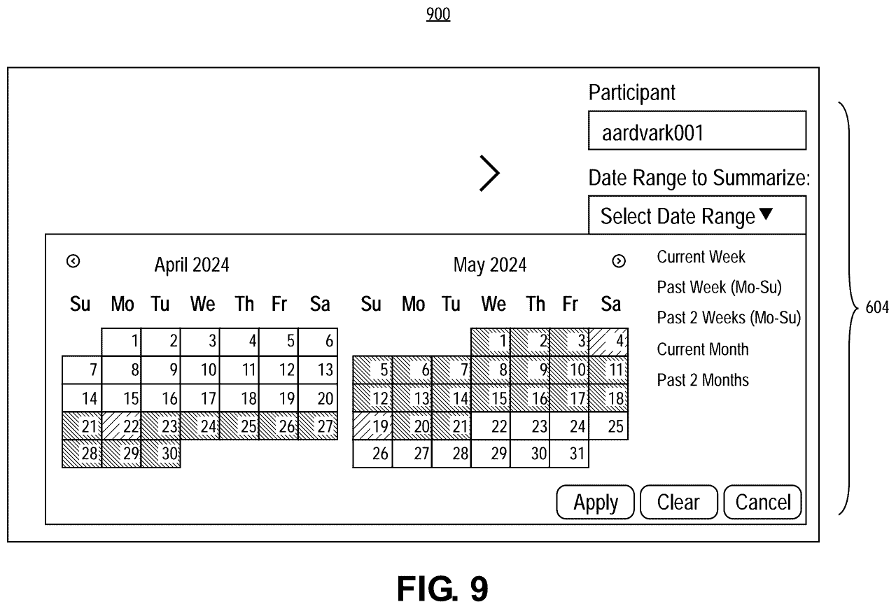

In some examples, the clinician 132 may be able to review the trends summary 601 via example patient report 600 in FIG. 6. The trends summary 601 may be a written summary, or it may be set to be read aloud to the clinician 132 (e.g., via a speaker on the clinician device 130, etc.). In some examples, the trends summary 601 may be divided into broader categories (e.g., trends, themes, events, etc.) and/or into more specific categories (e.g., mood, stress, social functioning, etc.). The trends summary 601 may be set to show certain categories by default or the clinician 132 may select which categories they want to view. The graph 602 may be a visual representation of the trends 603 of a patient's 162 condition. In some aspects, there may be a plurality of graphs 602. In the example patient report 600, the trends 603 are shown to trend either in a positive or negative direction or appear to be stable. The trends 603 are displayed on the graph 602, but also may displayed in a written form. The clinician 132 may be able to select parameters for the example patient report 600 via the parameter selection menu 604. The parameter selection menu 604 may include parameters, such as: patient, date ranges, type of generative AI algorithm 126, specificity of the report, etc. The example date selector 900 of FIG. 9 shows an example of how a clinician 132 may select the dates of the data entries they wish to view, such as by: selecting specific dates, selecting a date range, selecting preset ranges (for example “the last two weeks”), etc. The example patient report 600 and/or date selector 900 may be displayed on the clinician device 130. The example patient report 600 may also be stored on the clinician device 130 and/or the database 118.

To illustrate, in one exemplary implementation of the example of FIG. 2, after obtaining patient survey data 172 representing the patient's condition (block 230), determining trends 603 in the data may be done via the determination algorithm 124 (block 240). The determination algorithm 124 may analyze various entries and, based on their totals, assign a value. That value, in turn, may further be used to represent levels of severity of distress over time, such as: no stress, mild stress, moderate stress, severe stress, extreme stress, etc. A trend may form when the determination algorithm 124 determines that there is an acute change in the patient's condition over a short time, a gradual change in the patient's condition over the course of multiple concurrent entries, and/or if a patient's condition remains the same for multiple entries along with other similar variations. In another example, the determination algorithm 124 may instead analyze individual entries and assign them a value. That value, in turn, may be further used to represent various types of mental distress, such as: no depression, is stressed, is anxious, etc. An instance of both of the prior examples working together may be a result, such as: no stress, moderate depression, mild anxiety, etc.

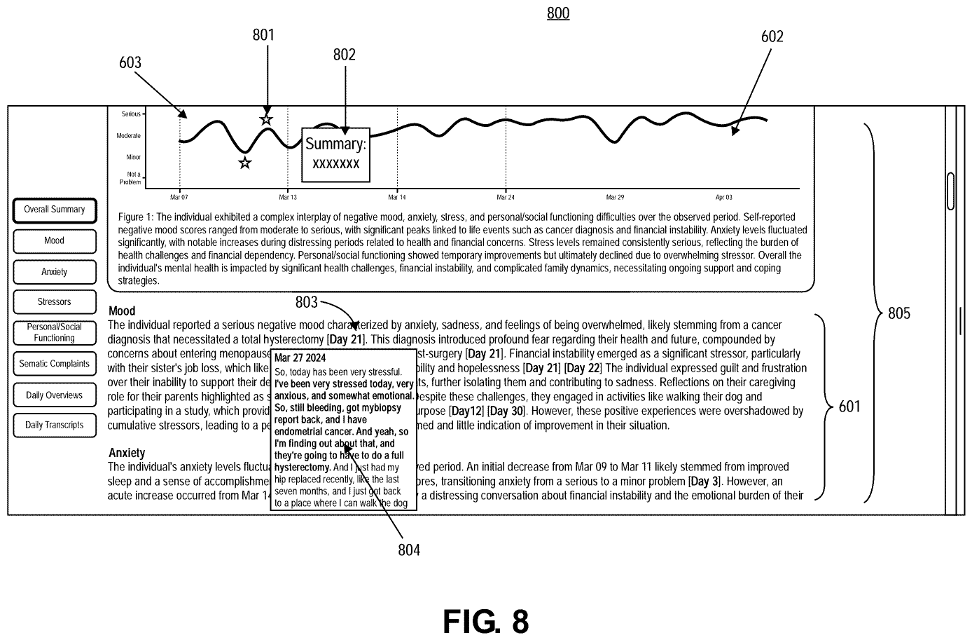

At block 250, a citation, such as citation 803 illustrated in the example of FIG. 8, may be determined. The citation 803 may connect the trends 603 to a portion of the transcription of the patient input data. After trends 603 have been determined at block 240, citations 803 to the transcription of block 220 may be created in order to allow for a reference to what in the transcription corresponds to the particular trend, thus explaining the trend. In some embodiments, the citation 803 may refer back to the patient input data 171 and/or patient survey data 172. Additionally or alternatively, the generative AI algorithm 126 may be applied to the citation 803 in order to create a summary of the events that caused the trends 603. That summary may further be linked to the transcription of block 220, thereby connecting a portion of the transcription to the particular trend.

In some embodiments, the example patient report 800 may include a marker 801 on the graph 602 as shown in FIG. 8. The marker 801 may indicate significant variability in the patient's 162 condition. The marker 801 may be a star, circle, cross, flag, etc. In some aspects, if the clinician 132 interacts with the marker 801 via clicking, hovering over with a mouse, etc., then an annotation box 802 may appear. The annotation box 802 may display a summary of likely causes and/or explanations of the significant variability denoted by the marker 801. Additionally or alternatively, the citations 803 may appear within the trends summary 601. These citations 803 may be specific references in the trends summary 601 to the patient input data 171 that was used to generate that portion of the trends summary 601. The clinician 132 may interact with the citations 803 by clicking, hovering, etc., in order to produce a citation display 804. The citation display 804 shows a transcript of the patient input data 171 that was used to generate the portion of the trends summary 601 that was cited.

Furthermore, the example method 200 may determine significant variability in the patient's condition (block 260). There may be multiple ways to determine significant variability, such as when a patient's condition jumps multiple steps from one entry to another. An example of significant variability may be when a patient's entry “A” is evaluated at block 240 to be “no stress” but a patient's entry “B” is evaluated as “severe stress.” It should be appreciated that the previous example illustrated a jump of three steps on the example scale (e.g., no stress, mild stress, moderate stress, severe stress, extreme stress). A significant variability may be set to be determined by any level of increase or decrease in any type of scale. As another example, going from a patient's entry “A” of “severe stress” to a patient's entry “B” as “Mild Stress” may been seen as significant variability. What is determined as a significant variability may be set by the clinician 132.

As part of the example method 200, the method may generate a graph 602 of the patient's condition (block 270). The graph 602 may be a line graph or any other type of graph (bar graph, pie chart, histogram, scatter plot, area charts, etc.). The graph 602 may show the patient's condition from day to day. Significant variability that was determined at block 260 may be especially noted on the graph, represented by a marker, for example, a star that signifies an important change. Furthermore, the user (e.g., the clinician 132, etc.) may be able to interact with the graph 602 (e.g., via the clinician device 130, etc.), such as by clicking, hovering or otherwise engaging with the graph 602 to display annotations (block 280) in order to determine what occurred on certain days or what is the cause of a significant variability.

The example method 200 may apply a generative AI algorithm to the patient input data 171, the patient survey data 172, the trends 603 in the patient survey data 172, or other data associated with the example method 200 in order to produce a report 805 of the trends of the patient's condition (block 290). The generative AI may be trained to cross reference the patient survey data 172 with the patient input data 171 in order to create an accurate report 805 on the patient's condition.

In some implementations, at block 290, the patient survey data 172 may be parsed for information based on what conditions are sought to be assessed (e.g., mood, anxiety, depression, etc.). During this process, key statistical features over the course of the selected time period, including consistent trends, events, and patterns of variability may be identified. The trained generative AI algorithm 126 then references the key statistical features along with the patient input data 171 in order to summarize the patient input data 171 in order to contextualize and explain the trends 603 and events of the patient's condition. Current methods of using generative AI to summarize patient input data do not reference patient survey data and, as a result, provide only summaries that often lack a way of measuring “severity” that can provide important context in creating an overall picture of a patient's condition over time.

In some embodiments, the report 805 may be an overview of the patient's entire condition. The report 805 may be further expanded to include a summary of the patient's condition with respect to their specific condition(s) or mood(s). For example, the expanded report 805 may include a summary of the patient's negative mood and/or a summary of the patient's anxiety. Furthermore, this report 805 may be additionally expanded to include a more detailed report 805 on the patient's specific condition or mood, which includes both an overview and a summary of that condition or mood.

In some implementations, the report 805 may include an overview of the patient's entire condition between their last visit and the time the report is generated (e.g., the current moment in time). The report 805 may further show summaries of the patient's negative mood, anxiety, stressors, and social functioning. In some embodiments, upon request, a more in-depth report 805 on the patient's stressors may be generated which may include an overview on the specifics and a more detailed summary of the patient's stressors is presented.

Example method 200, at block 295, may display the report 805 along with citations 803 from block 250, graphs 602 from block 270, and/or annotations from block 280 (e.g., on a display of the clinician device 130, etc.). Examples displays are illustrated by FIGS. 4-8.

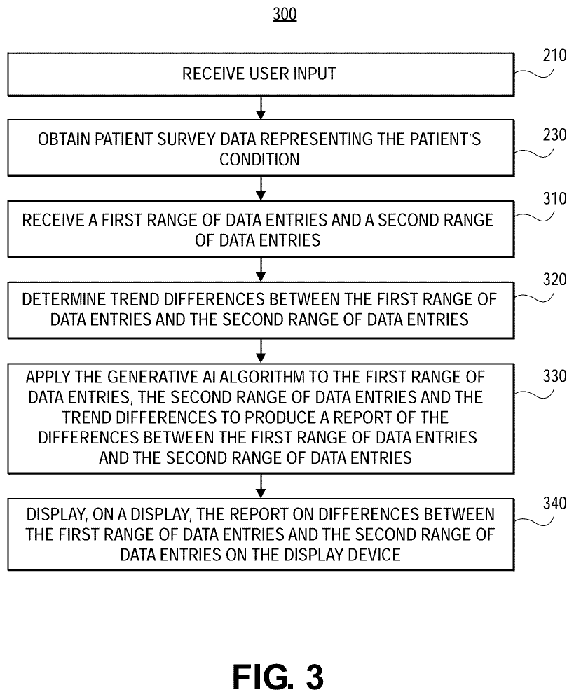

FIG. 3 shows an example method 300 of presenting an interval report of a patient's 162 conditions. For illustrative purposes, the following discussion will refer to the one or more processors 120 of the computing device 102 as performing the blocks of the example method 300. However, it should be understood that any other component (e.g., one or more processors of any of the data sources 170, one or more processors of the clinician device 130, one or more processors of the patient device 160, etc.) may perform any of the blocks instead of and/or in conjunction with the one or more processors 120.

Example method 300 may perform blocks 210 and 230 as described above. The clinician 132 may request that a report be generated in order to compare two intervals from the data source 170. This may be initiated by interacting with the interval change indicator 701. The one or more processors 120 may receive at least two sets of references, for example, two date ranges (block 310). It should be understood that while for illustrative purposes there are two intervals or date ranges, there may be any number of intervals and/or date ranges.

In example method 300, each set of date ranges may be evaluated for trends using a determination algorithm 124 similar to block 240. After which, the set of date ranges may be compared with each other to determine trend differences between the first range of data entries and the second range of data entries (block 320). This process may involve using a determination algorithm 124 in order to statistically compare the different sets of data in regards to the patient's 162 condition.

Furthermore, in example method 300, a generative AI algorithm 126 may be applied to the first range of data entries, the second range of data entries, and the trend differences to produce a report 805 of the differences between the data entries (block 330). The report 805 may summarize and/or explain detail: the patient's 162 conditions during some or all of the intervals, how the patient's condition has changed between intervals, and/or the likely causes of those changes.

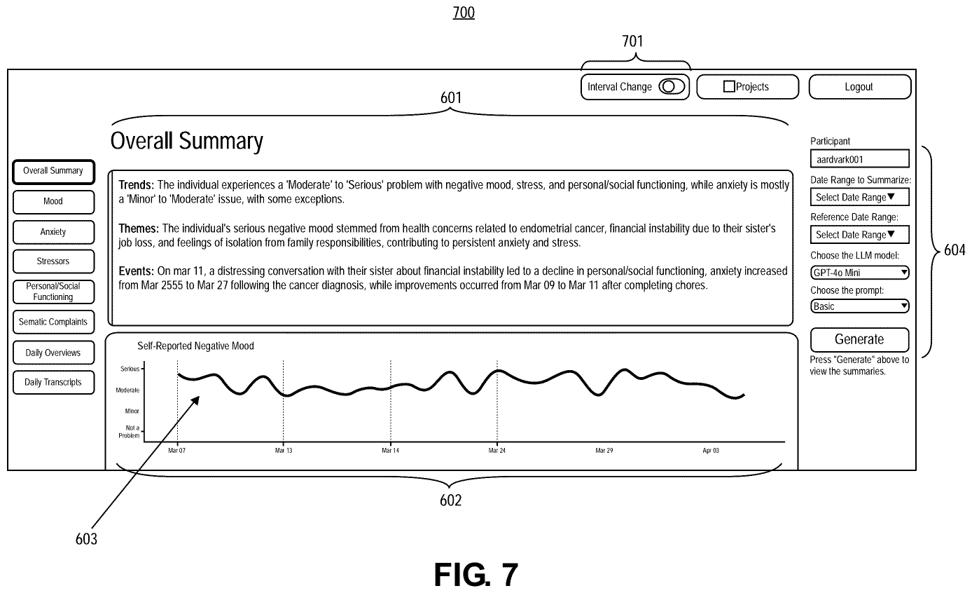

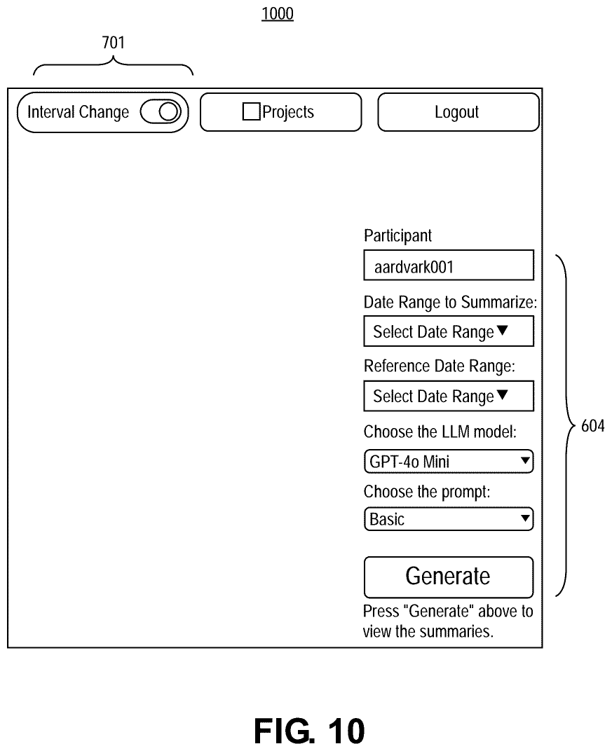

In FIG. 7, the example report 700 has an interval change indicator 701. The interval change indicator 701 allows the clinician 132 to interact with the indicator in order to switch to an interval change mode. The interval change indicator 701 may be a button, toggle, text, or any other interactable medium that when interacted with will allow the clinician 132 to generate a report 805 with interval change data. When interval change is selected, the parameter selection menu 604 may be updated to include additional parameters such as number of intervals, additional parameters for those number of intervals, etc. Example parameter selection menu 1000 in FIG. 10 shows an example of the parameter selection menu 604 updated to include additional parameters for an interval change report.

At block 340, the report 805, the citations 803, the graphs 602, and/or the annotations may be displayed (e.g., on a display of the clinician device 130, etc.). Examples displays are illustrated by FIGS. 4-8.

Furthermore a treatment may be administered based on the displayed report or any other information displayed at block 340. For example, the clinician 132 may determine, based on the displayed information, a particular type of medication to prescribe, which the patient 162 may subsequently take. In another example, the clinician 132 may determine, based on the displayed information, to increase or decrease the frequency of the patient's 162 visits with the clinician 162, and the patient may then visit the clinician (e.g., for therapy) based upon the changed frequency.

It should be understood that not all blocks and/or events of the exemplary signal diagrams and/or flowcharts are required to be performed. Moreover, the exemplary flowcharts are not mutually exclusive (e.g., block(s)/events from each example flowchart may be performed in any other flowchart). The exemplary flowcharts may include additional, less, or alternate functionality, including that discussed elsewhere herein.

Moreover, advantageously, the embodiments described above with respect to the examples of FIGS. 2 and 3 address a challenge often faced by clinician. That is, during periodic therapy sessions, patients often emphasize recent events and deemphasize (or even forget to mention) less recent events. By periodically gathering information from the patient, and then using the generative AI in accordance with the techniques described herein, the clinician is provided with an unbiased (or less biased) report of the patient's condition or trends in condition.

### Exemplary Improved Computer Security

Advantageously, some embodiments improve computer security of the system.

In some embodiments, both patients 162 and clinicians 132 use their respective patient device 160 or clinician device 132 to navigate to a website using a secure HTTPS connection. Advantageously, this improves computer security by ensuring that all data transmitted between the patient device 160 or clinician device 132 and the servers is encrypted.

In some examples, users (e.g., patients and clinicians) are authenticated as a security control mechanism. Users may not have the permission to register online; usernames and passwords may only be generated by application administrators. In some aspects, inputs may be checked for acceptable data types and validated to prevent injection attacks. Additionally, outputs may be sanitized to ensure that error messages, stack traces or memory dumps are not revealed. Computer security may be further improved by secure session management practices which may be enforced, including setting short session timeouts, automatically logging users out after a period of inactivity, and invalidating sessions upon logout to prevent unauthorized access. Further advantageously, some or all data may not be cached or stored on either the patient device 160 or clinician device 130.

To even further improve computer security, in some embodiments, collecting audio data from patients may use an application programming interface (API), which operates within a secure browser, ensuring that data is only accessible to the application. Additionally, OS and browser security notifications may be utilized to ensure that patients are aware of microphone permissions and when audio data is being collected. In some examples, multiple indicators on the application's user interface (UI) display when the microphone is recording, how long it has been recording, and the audio input levels. Precautionary measures may be taken to remove the audio data from the device's memory as soon as the data is safely transmitted for storage; for example, an audio recording may be automatically deleted from the patient device 160 in response to the audio recording being uploaded to the clinician device 130 and/or the computing device 102.

In some implementations, to even further improve computer security, on the clinician device 130, the generated reports 805 may only be rendered within the secure browser. The use of the HTTPS protocol may be used to ensure encrypted data is transmitted between the server and the clinician device 130. Additionally, by rendering reports 805 directly in the browser, the report 805, optionally, does not need to be stored on the clinician device 130.

In some embodiments, a virtual private cloud (VPC) may be setup and designed to improve data security and secure processing of sensitive information. The architecture may, in some examples, include a public subnet containing an Application Load Balancer (ALB) and a private subnet housing virtual machine instances and/or a relational database service (RDS) instance.

In some examples, there may be a public subnet with an application load balancer (ALB). Traffic between the user's device and the load balancer may be encrypted using the HTTPS protocol.

In other examples, there may be a private subnet with virtual machine and/or RDS instances. The virtual machine instances may run Apache servers that serve the web application. In some aspects the instances of virtual machine are isolated in the private subnet. The RDS instance may also exist in the private subnet and provide a database service for the web application that leverages security features such as encryption at rest and in transit, automated backups, and database snapshots. Security groups and Network ACLs may be configured to control inbound and outbound traffic to the virtual machine and RDS instances.

In further aspects, all data in transit to and from the VPC is encrypted using HTTPS with TLS. In some examples, the patient input data 171 or the patient survey data 172 collected from the patient device 160 does not flow into the VPC.

Further implementations may have the clinician device 130 use the virtual machine instances within the private subnet to generate reports 805. The report 805 generation algorithms may run on the virtual machine instances and pull sensitive data from the bucket (e.g., container) on-demand. The virtual machine instances may, in some instances, use a highly restricted identity and access management (IAM) role and a software development kit (SDK) to access the bucket. The SDK may provide secure and encrypted interactions with the service. The IAM role, in some examples, advantageously grants only the minimum permissions required to read data, ensuring that even if an instance is compromised, the scope of access is limited. Additionally, in some examples, no sensitive data is ever stored on the virtual machine instance store, EBS, or EFS; and measures may be put into place to wipe all data from memory upon the successful or failed conclusion of each algorithm call.

Additional embodiments may generate the reports 805, by having the virtual machine instances also communicate using the generative AI API. In some examples, API requests are made over HTTPS, ensuring that data transmitted to and from the API servers is encrypted.

In some aspects, a survey manager may be employed to collect and store survey data from the patients 162. The patients 162, in some cases, complete a one-time onboarding survey and daily surveys. These surveys may be hosted by the survey manager, and the data collected may be stored exclusively by the survey manager and encrypted at rest.

Furthermore, advantageously, in some embodiments, the data stored by the survey manager may only be accessed by processes running on a Health Information Technology and Services (HITS) managed server behind a firewall. This data may be accessed using a survey manager API, which could ensure that all data transmitted between the survey manager and the HITS managed server is encrypted in transit.

Additional embodiments may use a particular service which provides comprehensive data security through multiple mechanisms. Server-side encryption using AES-256 ensures that data at rest is protected. In some examples, access control mechanisms may be utilized through IAM policies, bucket policies, and Access Control Lists (ACLs) to ensure that the storage is not publicly accessible, and that access is finely controlled for authorized users and applications. This may ensure that only specific, authenticated entities from trusted origins can read the data; and only requests originating from trusted domains are allowed to write data to the bucket.

In certain implementations, the patient device 160 may be configured to allow for audio data collected from the verbalized entries to be directly uploaded from the patient device 160 to a bucket using the SDK. This data is encrypted in transit and remains encrypted at rest. As an additional measure of safety, every four hours, this data is moved behind a firewall onto HITS-managed storage using the SDK. The audio data may then be deleted from the bucket.

In certain implementations, the clinician device 130 may be configured to allow for, transcriptions of entries stored behind the firewall are uploaded daily from a HITS-managed server (e.g., the computing device 102, etc.) onto the bucket using the SDK. When prompted by the clinician 132, this data may be accessed by the report-generating algorithms running on virtual machine instances in the virtual private cloud (VPC) using the SDK for further processing.

In further aspects, a cron job running on the HITS managed server may check the bucket for new audio recording data at set intervals. It may copy this data onto the HITS managed storage and delete it from the bucket. Following this, a local audio transcription model may be launched to convert the new audio recording data into text transcripts. These transcripts may subsequently be uploaded to the bucket. Read, write, and delete operations between this server and the bucket may be performed using the SDK and tightly secured IAM credentials.

Additionally, cron jobs running on this server may periodically retrieve patient 162 contact information from the survey mangers to send notifications (reminders, automated data quality flags, etc.) using various notification APIs.

In some aspects, the data source 170 is populated manually and stores sensitive demographic, contact, consent, and other similar data.

In various examples, the patient 162 may enroll in daily automated text or email reminders to complete their entries. This enrollment may occur during an onboarding survey, but patients 162 are free to modify their opt-in preferences at any time. Patients 162 may also opt-in to receive notifications about payments and automated data quality checks.

Additionally, automated text messages may be sent by a process running on the HITS managed server using a notification API.

### Exemplary Training of an Exemplary Chatbot

The generative AI algorithm 126 (e.g., a chatbot, etc.) may, inter alia: (i) produce the report 805, and (ii) provide tailored, conversational-like services (e.g., answering clinician 132 questions, etc.). The generative AI algorithm 126 may be capable of understanding requests, providing relevant information, escalating issues, etc. Additionally, the generative AI algorithm 126 may generate data from interactions which the enterprise may use to personalize future support and/or improve the chatbot's functionality, e.g., when retraining and/or fine-tuning the chatbot. Moreover, although the following discussion may refer to an ML chatbot or an ML model, it should be understood that it applies equally to an AI chatbot or an AI model.

The generative AI algorithm 126 may be trained by generative AI training application 128 using large training datasets of text which may provide sophisticated capability for natural-language tasks, such as answering questions and/or holding conversations. The generative AI algorithm 126 may include a general-purpose pretrained LLM which, when provided with a starting set of words and/or patient survey data (prompt) as an input, may attempt to provide an output (response) of the most likely set of words that follow from the input (e.g., an explanation of the patient's condition). In one aspect, the prompt may be provided to, and/or the response received from, the generative AI algorithm 126 and/or any other ML model, via display 129, a display of the clinician device 130, and/or a display of the patient device 160. This may include a user interface device operably connected via an I/O module. Exemplary user interface devices may include a touchscreen, a keyboard, a mouse, a microphone, a speaker, a display, and/or any other suitable user interface devices.

Multi-turn (i.e., back-and-forth) conversations may require LLMs to maintain context and coherence across multiple user utterances, which may require the generative AI algorithm 126 to keep track of an entire conversation history as well as the current state of the conversation. The generative AI algorithm 126 may rely on various techniques to engage in conversations with users, which may include the use of short-term and long-term memory. Short-term memory may temporarily store information (e.g., in the memory 122 of the computing device 102) that may be required for immediate use and may keep track of the current state of the conversation and/or to understand the user's latest input in order to generate an appropriate response. Long-term memory may include persistent storage of information (e.g., the database 118 of the computing device 102) which may be accessed over an extended period of time. The long-term memory may be used by the generative AI algorithm 126 to store information about the user (e.g., preferences, chat history, etc.) and may be useful for improving an overall user experience by enabling the generative AI algorithm 126 to personalize and/or provide more informed responses.

In some embodiments, the system and methods to generate and/or train an ML chatbot model (e.g., via the generative AI training application 128) which may be used in the generative AI algorithm 126, may include three steps: (1) a supervised fine-tuning (SFT) step where a pretrained language model (e.g., an LLM) may be fine-tuned on a relatively small amount of demonstration data curated by human labelers to learn a supervised policy (SFT ML model) which may generate responses/outputs from a selected list of prompts/inputs. The SFT ML model may represent a cursory model for what may be later developed and/or configured as the ML chatbot model; (2) a reward model step where human labelers may rank numerous SFT ML model responses to evaluate the responses which best mimic preferred human responses, thereby generating comparison data. The reward model may be trained on the comparison data; and/or (3) a policy optimization step in which the reward model may further fine-tune and improve the SFT ML model. The outcome of this step may be the ML chatbot model using an optimized policy. In one aspect, step one may take place only once, while steps two and three may be iterated continuously, e.g., more comparison data is collected on the current ML chatbot model, which may be used to optimize/update the reward model and/or further optimize/update the policy.

### Supervised Fine-Tuning ML Model

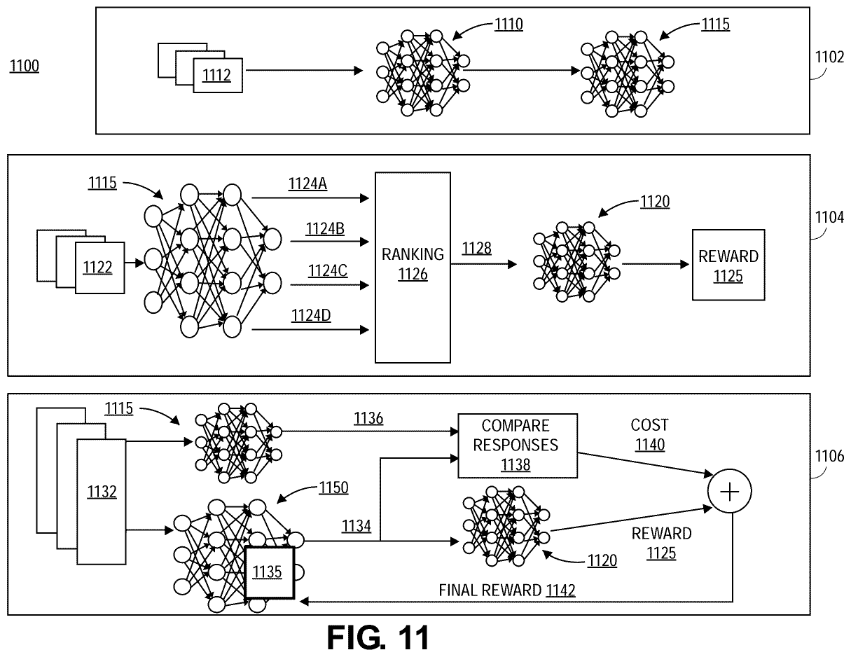

As an initial matter, although the discussion with respect to FIG. 11 refers to ML model 1150, it should be understood that 1150 may refer equally to an AI and/or ML algorithm and/or model.

FIG. 11 depicts a combined block and logic diagram 1100 for training an ML chatbot model, in which the techniques described herein may be implemented, according to some embodiments. It should be understood that FIG. 11 may apply to training any generative AI and/or ML algorithm, and/or chatbot described herein, and FIG. 11 should not be considered to be restricted to the generative AI algorithm 126. In addition, the generative AI algorithm 126 may be trained in accordance with any of the other techniques described herein; and the training of generative AI algorithm 126 should not be considered restricted to the teachings of FIG. 11.

Some of the blocks in FIG. 11 may represent hardware and/or software components, other blocks may represent data structures or memory storing these data structures, registers, or state variables (e.g., 1112), and other blocks may represent output data (e.g., 1125). Input and/or output signals may be represented by arrows labeled with corresponding signal names and/or other identifiers. The methods and systems may include one or more blocks 1102, 1104, 1106, which will be described in further detail below. In some embodiments, any or all of the blocks 1102, 1104, 1106 are servers.

In one aspect, at block 1102, a pretrained language model 1110 may be fine-tuned. The pretrained language model 1110 may be obtained at block 1102 and be stored in a memory, such as memory 122 and/or database 118. The pretrained language model 1110 may be loaded into an ML training module at block 1102 for retraining/fine-tuning. A supervised training dataset 1112 may be used to fine-tune the pretrained language model 1110 wherein each data input prompt to the pretrained language model 1110 may have a known output response for the pretrained language model 1110 to learn from. The supervised training dataset 1112 may be stored in a memory at block 1102, e.g., the memory 122 or the database 118. In one aspect, the data labelers may create the supervised training dataset 1112 prompts and appropriate responses. The pretrained language model 1110 may be fine-tuned using the supervised training dataset 1112 resulting in the SFT ML model 1115 which may provide appropriate responses to user prompts once trained. The trained SFT ML model 1115 may be stored in a memory, such as the memory 122 or the database 118.

In one aspect, the supervised training dataset 1112 may include prompts (e.g., patient input data, patient survey data, trends in the survey data determined by the determination algorithm 124, etc.) and responses (e.g., reports 805, etc.) which may be relevant to clinician 132 and/or patient 162. An example of a prompt includes: (i) patient input data 171, (ii) patient survey data 172, and a trend 603. An example of a response includes report 805. Examples of the patient input data 171, patient survey data 172, trend 603 and report 805 are described elsewhere herein (e.g., report 805 may include citation 803, etc.).

### Training the Reward Model

In one aspect, training the ML chatbot model 1150 may include, at block 1104, training a reward model 1120 to provide as an output a scaler value/reward 1125. The reward model 1120 may be required to leverage Reinforcement Learning with Human Feedback (RLHF) in which a model (e.g., ML chatbot model 1150) learns to produce outputs which maximize its reward 1125, and in doing so may provide responses which are better aligned to user prompts.

Training the reward model 1120 may include, at block 1104, providing a single prompt 1122 to the SFT ML model 1115 as an input. The input prompt 1122 may be provided via an input device (e.g., a keyboard) of the computing device 102. The prompt 1122 may be previously unknown to the SFT ML model 1115, e.g., the labelers may generate new prompt data, the prompt 1122 may include testing data stored on database 118, data source 170, and/or any other suitable prompt data. The SFT ML model 1115 may generate multiple, different output responses 1124A, 1124B, 1124C, 1124D to the single prompt 1122. At block 1104, the computing device 102 (and/or the clinician device 130, patient device 160, etc.) may output the responses 1124A, 1124B, 1124C, 1124D via any suitable technique, such as outputting via a display (e.g., as text responses), a speaker (e.g., as audio/voice responses), etc., for review by the data labelers.

The data labelers may provide feedback (e.g., via the computing device 102, the clinician device 130, the patient device 160, etc.) on the responses 1124A, 1124B, 1124C, 1124D when ranking 1126 them from best to worst based upon the prompt-response pairs. The data labelers may rank 1126 the responses 1124A, 1124B, 1124C, 1124D by labeling the associated data. The ranked prompt-response pairs 1128 may be used to train the reward model 1120. In one aspect, the computing device 102 may load the reward model 1120 via the generative AI training application 128 and train the reward model 1120 using the ranked response pairs 1128 as input. The reward model 1120 may provide as an output the scalar reward 1125.

In one aspect, the scalar reward 1125 may include a value numerically representing a human preference for the best and/or most expected response to a prompt, i.e., a higher scaler reward value may indicate the user is more likely to prefer that response, and a lower scalar reward may indicate that the user is less likely to prefer that response. For example, inputting the “winning” prompt-response (i.e., input-output) pair data to the reward model 1120 may generate a winning reward. Inputting a “losing” prompt-response pair data to the same reward model 1120 may generate a losing reward. The reward model 1120 and/or scalar reward 1136 may be updated based upon labelers ranking 1126 additional prompt-response pairs generated in response to additional prompts 1122.

In one example, a data labeler may provide to the SFT ML model 1115 as an input prompt 1122, “Describe the sky.” The input may be provided by the labeler (e.g., via the computing device 102, etc.) to the computing device 102 running generative AI algorithm 126 utilizing the SFT ML model 1115. The SFT ML model 1115 may provide as output responses to the labeler (e.g., via their respective devices): (i) “the sky is above” 1124A; (ii) “the sky includes the atmosphere and may be considered a place between the ground and outer space” 1124B; and (iii) “the sky is heavenly” 1124C. The data labeler may rank 1126, via labeling the prompt-response pairs, prompt-response pair 1122/1124B as the most preferred answer; prompt-response pair 1122/1124A as a less preferred answer; and prompt-response 1122/1124C as the least preferred answer. The labeler may rank 1126 the prompt-response pair data in any suitable manner. The ranked prompt-response pairs 1128 may be provided to the reward model 1120 to generate the scalar reward 1125. It should be appreciated that this facilitates training the generative AI algorithm 126 to determine questions corresponding various types of insurance policies, and answers corresponding to the types of insurance policies.

While the reward model 1120 may provide the scalar reward 1125 as an output, the reward model 1120 may not generate a response (e.g., text). Rather, the scalar reward 1125 may be used by a version of the SFT ML model 1115 to generate more accurate responses to prompts, i.e., the SFT model 1115 may generate the response such as text to the prompt, and the reward model 1120 may receive the response to generate a scalar reward 1125 of how well humans perceive it. Reinforcement learning may optimize the SFT model 1115 with respect to the reward model 1120 which may realize the configured ML chatbot model 1150.

### RLHF to Train the ML Chatbot Model

In one aspect, the computing device 102 may train the ML chatbot model 1150 (e.g., via the generative AI training application 128) to generate a response 1134 to a random, new and/or previously unknown user prompt 1132. To generate the response 1134, the ML chatbot model 1150 may use a policy 1135 (e.g., algorithm) which it learns during training of the reward model 1120, and in doing so may advance from the SFT model 1115 to the ML chatbot model 1150. The policy 1135 may represent a strategy that the ML chatbot model 1150 learns to maximize its reward 1125. As discussed herein, based upon prompt-response pairs, a human labeler may continuously provide feedback to assist in determining how well the ML chatbot's 1150 responses match expected responses to determine rewards 1125. The rewards 1125 may feed back into the ML chatbot model 1150 to evolve the policy 1135. Thus, the policy 1135 may adjust the parameters of the ML chatbot model 1150 based upon the rewards 1125 it receives for generating good responses. The policy 1135 may update as the ML chatbot model 1150 provides responses 1134 to additional prompts 1132.

In one aspect, the response 1134 of the ML chatbot model 1150 using the policy 1135 based upon the reward 1125 may be compared using a cost function 1138 to the SFT ML model 1115 (which may not use a policy) response 1136 of the same prompt 1132. The server 1106 may compute a cost 1140 based upon the cost function 1138 of the responses 1134, 1136. The cost 1140 may reduce the distance between the responses 1134, 1136, i.e., a statistical distance measuring how one probability distribution is different from a second, in one aspect the response 1134 of the ML chatbot model 1150 versus the response 1136 of the SFT model 1115. Using the cost 1140 to reduce the distance between the responses 1134, 1136 may avoid a server over-optimizing the reward model 1120 and deviating too drastically from the human-intended/preferred response. Without the cost 1140, the ML chatbot model 1150 optimizations may result in generating responses 1134 which are unreasonable but may still result in the reward model 1120 outputting a high reward 1125.

In one aspect, the responses 1134 of the ML chatbot model 1150 using the current policy 1135 may be passed by the server 1106 to the rewards model 1120, which may return the scalar reward or discount 1125. The ML chatbot model 1150 response 1134 may be compared via cost function 1138 to the SFT ML model 1115 response 1136 by the server 1106 to compute the cost 1140. The server 1106 may generate a final reward 1142 which may include the scalar reward 1125 offset and/or restricted by the cost 1140. The final reward or discount 1142 may be provided by the server 1106 to the ML chatbot model 1150 and may update the policy 1135, which in turn may improve the functionality of the ML chatbot model 1150.

To optimize the ML chatbot model 1150 over time, RLHF via the human labeler feedback may continue ranking 1126 responses of the ML chatbot model 1150 versus outputs of earlier/other versions of the SFT ML model 1115, i.e., providing positive or negative rewards 1125. The RLHF may allow the generative AI training application 128 to continue iteratively updating the reward model 1120 and/or the policy 1135. As a result, the ML chatbot model 1150 may be retrained and/or fine-tuned based upon the human feedback via the RLHF process, and throughout continuing conversations may become increasingly efficient.

Although multiple blocks 1102, 1104, 1106 are depicted in the exemplary block and logic diagram 1100, each providing one of the three steps of the overall ML chatbot model 1150 training, fewer and/or additional blocks/servers may be utilized and/or may provide the one or more steps of the generative AI algorithm 126 training. In one aspect, one server may provide the entire ML chatbot model 1150 training.

### Exemplary Validation and Ranking

Advantageously, at block 1104, the following example illustrates how a questionnaire may be used to improve accuracy of the system.

For validation, in one example 72 subjects were recruited with mild to moderate depression/anxiety, and each provided 30 days of voice diaries. A subset of 20 of these subjects provided an additional 30 days. From these approximately 2700 voice diaries, clinical reports were generated according to the techniques described herein.

Subsequently, a validation study was performed. Eight human experts (research assistants) used a structured rubric to evaluate the clinical reports generated by the app. The rubric asked yes/no questions about display elements of the clinical reports in terms of accuracy, informativeness, conciseness, and other dimensions. These may be time consuming assessments because the experts have to go back to the primary day-by-day transcripts to verify accuracy and informativeness given the context, etc. Experts took about three hours for each assessment.

TABLE 1
Below illustrates the results.

| | AI- | Human- |
|---|---|---|
| | Generated | Generated |
| Theme Selection Results | Summaries | Summaries |
| Is the title informative and helpful? | 98% | 79% |
| Is the expansion accurate? | 98% | N/A |
| Is the expansion informative and | 98% | N/A |
| helpful? | | |
| Is the example accurate? | 92% | 81% |
| Is the example informative and helpful? | 90% | 73% |
| Are all important examples present? | 93% | 41% |
| How may important examples were | 0.15 | 1.0 |
| missed? | | |
| Overall, is the THEMES section | 98% | 81% |
| informative and helpful? | | |

Experts also generated clinical summaries from the day-by-day transcripts themselves. This serves as an additional check: It allows to assess how app-generated summaries compare to human-generated summaries. Generating these summaries takes approximately 3-5 hours each. Some elements may optionally be left off (such as citations for key claims in the patient's own words) if it is determined they take too much time for humans to do.

### Other Matters

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations). A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Upon reading this disclosure, those of skill in the art will appreciate still additional alternative structural and functional designs for the approaches described herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

Furthermore, the patent claims at the end of this patent application are not intended to be construed under 35 U.S.C. § 112 (f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed:

1. A computer-implemented method for presenting a report of a patient's condition using a generative artificial intelligence (AI) algorithm, the computer-implemented method comprising:
receiving, by one or more processors, patient input data;
obtaining, by the one or more processors, patient survey data representing the patient's condition;
determining, by the one or more processors, a trend in the patient survey data using a determination algorithm;
applying, by the one or more processors, the generative AI algorithm to the patient input data, the patient survey data, and the trend in the patient survey data to produce a report of the trend of the patient's condition; and
displaying, by the one or more processors, the report on a display device.

2. The computer-implemented method of claim 1, wherein the patient input data is a voice recording.

3. The computer-implemented method of claim 2, further comprising:
applying, by the one or more processors, a natural language processor to the voice recording to produce a transcription of the voice recording.

4. The computer-implemented method of claim 1, further comprising:
determining, by the one or more processors, a citation connecting the trend to a portion of the patient input data; and
displaying, by the one or more processors, the citation on the display device.

5. The computer-implemented method of claim 4, further comprising:
determining, by the one or more processors, a significant variability in the patient's condition; and
displaying, by the one or more processors, a graph of the patient's condition based on the patient survey data with a marker of the significant variability in the patient's condition.

6. The computer-implemented method of claim 5, further comprising:
determining, by the one or more processors, an annotation of details of events of the significant variability in the patient's condition; and
displaying, by the one or more processors, the annotation of details of events to a clinician when requested.

7. The computer-implemented method of claim 1, further comprising:
receiving, by the one or more processors, a first range of data entries and a second range of data entries;
determining, by the one or more processors, statistical differences between the first range of data entries and the second range of data entries;
applying, by the one or more processors, the generative AI algorithm to the first range of data entries, the second range of data entries, and the statistical differences to produce a report of the statistical differences between the first range of data entries and the second range of data entries; and
displaying, by the one or more processors, the report of the statistical differences between the first range of data entries and the second range of data entries on the display device.

8. A computer system for presenting a report of a patient's condition using a generative artificial intelligence (AI) algorithm, the computer system comprising one or more processors configured to:
receive patient input data;
obtain patient survey data representing the patient's condition;
determine a trend in the patient survey data using a determination algorithm;
apply the generative AI algorithm to the patient input data, the patient survey data, and the trend in the patient survey data to produce a report of the trend of the patient's condition; and
display the report on a display device.

9. The computer system of claim 8, wherein the report of the trend includes a text explanation of the trend.

10. The computer system of claim 8, wherein the patient input data comprises a voice recording, and wherein the one or more processors are further configured to:
apply a natural language processor to the voice recording to produce a transcription of the voice recording.

11. The computer system of claim 8, wherein the one or more processors are further configured to:
determine a citation connecting the trend to a portion of the patient input data; and
display the citation on the display device.

12. The computer system of claim 11, wherein the one or more processors are further configured to:
determine a significant variability in the patient's condition; and
display a graph of the patient's condition based on the patient survey data with a marker of the significant variability in the patient's condition.

13. The computer system of claim 12, wherein the one or more processors are further configured to:
determine an annotation of details of events of the significant variability in the patient's condition; and
display the annotation of details of events to a clinician when requested.

14. The computer system of claim 8, wherein the one or more processors are further configured to:
receive a first range of data entries and a second range of data entries;
determine statistical differences between the first range of data entries and the second range of data entries;
apply the generative AI algorithm to the first range of data entries, the second range of data entries, and the statistical differences to produce a report of the statistical differences between the first range of data entries and the second range of data entries; and
display the report of the statistical differences between the first range of data entries and the second range of data entries on the display device.

15. A computing device for presenting a report of a patient's condition using a generative artificial intelligence (AI) algorithm, the computing device comprising:
one or more processors; and
one or more memories;
the one or more memories having stored thereon computer-executable instructions that, when executed by the one or more processors, cause the computing device to:
receive patient input data;
obtain patient survey data representing the patient's condition;
determine a trend in the patient survey data using a determination algorithm;
apply the generative AI algorithm to the patient input data, the patient survey data, and the trend in the patient survey data to produce a report of the trend of the patient's condition; and
display the report on a display device.

16. The computing device of claim 15, wherein the patient input data comprises a voice recording.

17. The computing device of claim 15, the one or more memories having stored thereon computer executable instruction that, when executed by the one or more processors, cause the computing device to:
determine a citation connecting the trend to a portion of the patient input data; and
display the citation on the display device.

18. The computing device of claim 17, the one or more memories having stored thereon computer executable instruction that, when executed by the one or more processors, cause the computing device to:
determine a significant variability in the patient's condition; and
display a graph of the patient's condition based on the patient survey data with a marker of the significant variability in the patient's condition.

19. The computing device of claim 18, the one or more memories having stored thereon computer executable instruction that, when executed by the one or more processors, cause the computing device to:
determine an annotation of details of events of the significant variability in the patient's condition; and
display the annotation of details of events to a clinician when requested.

20. The computing device of claim 15, the one or more memories having stored thereon computer executable instruction that, when executed by the one or more processors, cause the computing device to:
receive a first range of data entries and a second range of data entries;
determine statistical differences between the first range of data entries and the second range of data entries;
apply the generative AI algorithm to the first range of data entries, the second range of data entries, and the statistical differences to produce a report of the statistical differences between the first range of data entries and the second range of data entries; and
display the report of the statistical differences between the first range of data entries and the second range of data entries on the display device.
